(19) United States
(12) Patent Application Publication
(10) Pub. No.: US 2015/0182139 A1
(43) Pub. Date: Jul. 2, 2015

(54) ELECTRONIC APPARATUS, METHOD AND STORAGE MEDIUM

(71) Applicant: Kabushiki Kaisha Toshiba, Tokyo (JP)

(72) Inventors: Yasuhiro Kanishima, Tokyo (JP); Takashi Sudo, Fuchu-shi (JP)

(21) Appl. No.: 14/326,940

(22) Filed: Jul. 9, 2014

(30) Foreign Application Priority Data
Dec. 26, 2013 (JP) 2013-268629

(51) Int. Cl. A61B 5/024; A61B 5/00

(52) U.S. Cl. CPC A61B 5/02444; A61B 5/681

(57) ABSTRACT

According to one embodiment, an electronic apparatus wearable by a user includes one or more sensors, a processor and a controller. The processor is configured to determine whether the user is in a waking state or a sleeping state by using a detected value of at least one sensor of the one or more sensors. The controller is configured to set an operation mode of the apparatus in a first apparatus mode if it is determined that the user is in a waking state, and to set the operation mode of the apparatus in a second apparatus mode if it is determined that the user is in a sleeping state.

### CROSS-REFERENCE TO RELATED APPLICATIONS

This application is based upon and claims the benefit of priority from Japanese Patent Application No. 2013-268629, filed Dec. 26, 2013, the entire contents of which are incorporated herein by reference.

### FIELD

Embodiments described herein relate generally to a low-power control technique suitable for a type of electronic apparatus that is worn on the human body like, for example, a wristwatch or a pair of glasses.

### BACKGROUND

In recent years, battery-operated, portable electronic apparatuses such as tablet terminals and smartphones have become widespread. Also, recently, a type of electronic apparatus that is called a wearable terminal, which is worn on the human body like a wristwatch or a pair of glasses, has also appeared.

Because wearable terminals are worn on the human body, they must be small and light and batteries for supplying electric power for operation also have limited capacity. Thus, reduction in power consumption by wearable terminals is strongly required.

### BRIEF DESCRIPTION OF THE DRAWINGS

A general architecture that implements the various features of the embodiments will now be described with reference to the drawings. The drawings and the associated descriptions are provided to illustrate the embodiments and not to limit the scope of the invention.

FIG. 1 is an exemplary perspective view showing the outside of an electronic apparatus according to an embodiment.

FIG. 2 is an exemplary illustration showing a system configuration of the electronic apparatus according to the embodiment.

FIG. 3 is an exemplary state transition diagram of a pulse sensor in the electronic apparatus according to the embodiment.

FIG. 4 is an exemplary functional block diagram related to reduction in power consumption by the pulse sensor of the electronic apparatus according to the embodiment.

FIG. 5 is an exemplary flowchart showing a procedure of processing of reduction in power consumption by the pulse sensor executed by the electronic apparatus according to the embodiment.

### DETAILED DESCRIPTION

Various embodiments will be described hereinafter with reference to the accompanying drawings.

In general, according to one embodiment, an electronic apparatus wearable by a user includes one or more sensors, a processor and a controller. The processor is configured to determine whether the user is in a waking state or a sleeping state by using a detected value of at least one sensor of the one or more sensors. The controller is configured to set an operation mode of the apparatus in a first apparatus mode if it is determined that the user is in a waking state, and to set the operation mode of the apparatus in a second apparatus mode if it is determined that the user is in a sleeping state.

An electronic apparatus according to the embodiment is implemented as a so-called wearable terminal, which is the type to be worn on the human body. Here, it is assumed that the electronic apparatus is implemented as a wearable terminal having the shape of a wristwatch and is permanently worn on an arm portion (wrist) of a user.

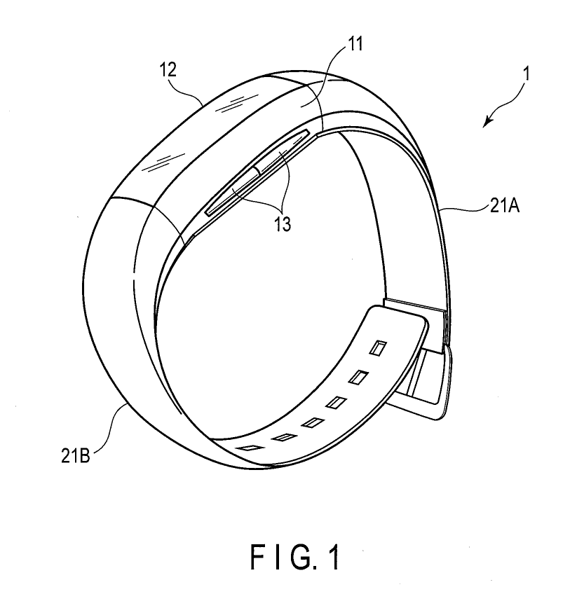

FIG. 1 is an exemplary perspective view of a wearable terminal 1. The wearable terminal 1 includes a main body 11. The main body 11 has a thin housing. In the housing, various electronic components are provided. On a top surface of the main body 11, a display 12 like a liquid crystal display (LCD) is disposed. The display 12 may be a touchscreen display which can detect a touch position on its display screen. On a side surface of the main body 11, an operation button 13 is disposed.

The wearable terminal 1 has belts (bands) 21A and 21B for wearing the main body 11 on the human body (arm portion). The belts 21A and 21B are each implemented with a component having flexibility.

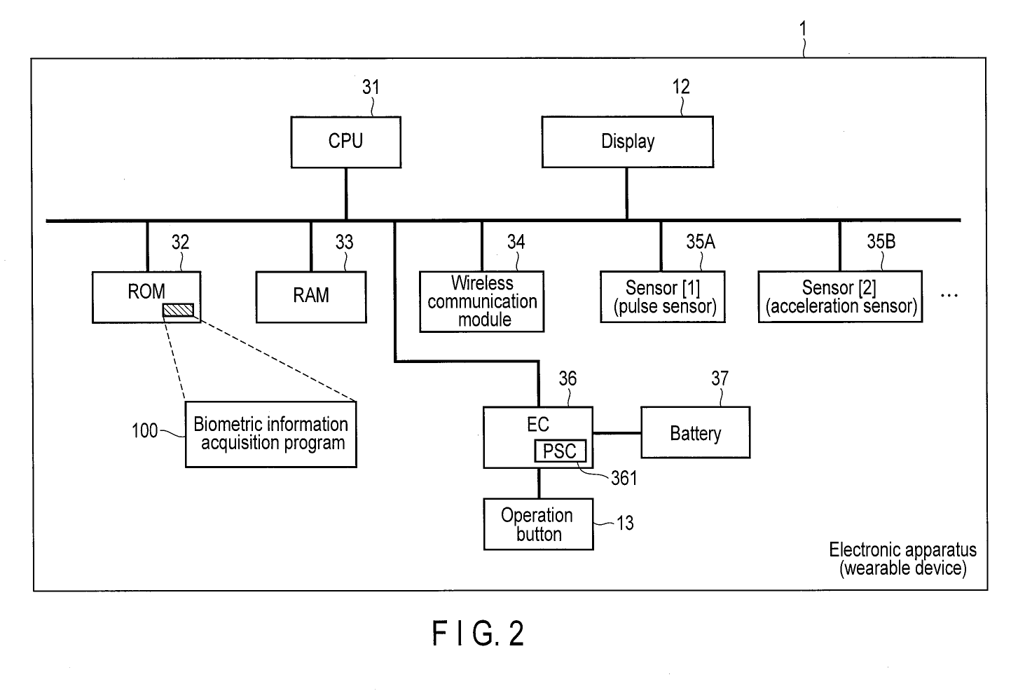

FIG. 2 is an exemplary illustration showing a system configuration of the wearable terminal 1.

As shown in FIG. 2, in addition to the display 12 and the operation button 13 shown in FIG. 1, a CPU 31, an ROM 32, an RAM 33, a wireless communication module 34, a plurality of sensors 35A, 35B, . . . , an embedded controller (EC) 36, a battery 37, and the like are disposed in the main body 11 of the wearable terminal 1.

The CPU 31 is a processor configured to control operations of various modules in the wearable terminal 1. The CPU 31 is configured to execute various programs stored in the ROM 32 using the RAM 33 as a working area. As one of the various programs, there is a biometric information acquisition program 100, which will be described later.

The wireless communication module 34 is a module configured to perform wireless communication conforming to, for example, the IEEE 802.11g standard. The plurality of sensors 35A, 35B, . . . , are, for example, a pulse sensor, an acceleration sensor, an angular velocity sensor, a geomagnetic sensor, a temperature sensor, a humidity sensor, and an illuminance sensor. Here, it is assumed that the sensor 35A is a pulse sensor and the sensor 35B is a three-axis acceleration sensor. A detected value of each sensor is stored in the RAM 33, and is used by the various programs including the biometric information acquisition program 100.

The EC 36 is a single-chip microcomputer including a power supply controller (PSC) 361 configured to administer supply control of electric power of the battery 37 to the various modules in the wearable terminal 1. The EC 36 includes a function of receiving an instruction from a user by an operation of the operation button 13.

The biometric information acquisition program 100 is a program configured to acquire biometric information such as a pulse and an active state of an autonomic nerve of the user wearing the wearable terminal 1 by means of the pulse sensor 35A, for example. The pulse sensor 35A is, for example, a reflective photoelectric sensor, and is configured to measure strength and weakness of bloodstream by receiving reflected light of light emitted to a blood vessel by means of a phenomenon in which hemoglobin in blood absorbs light. In the case of a transmission-type photoelectric sensor, light transmitted through a blood vessel is received. In any case, when bloodstream is strong, the amount of light absorbed by hemoglobin becomes large in comparison with that when bloodstream is weak, and thus the amount of received reflected light or received transmitted light becomes small.

The power consumption of the pulse sensor 35A configured to measure a pulse by emitting light in this manner accounts for a large proportion of the total power consumption of the wearable terminal 1. Thus, the wearable terminal 1 according to the embodiment includes a mechanism for reducing power consumption appropriately in accordance with circumstances controlling a light-emitting power of the pulse sensor 35A adaptively. This point will be hereinafter described in detail.

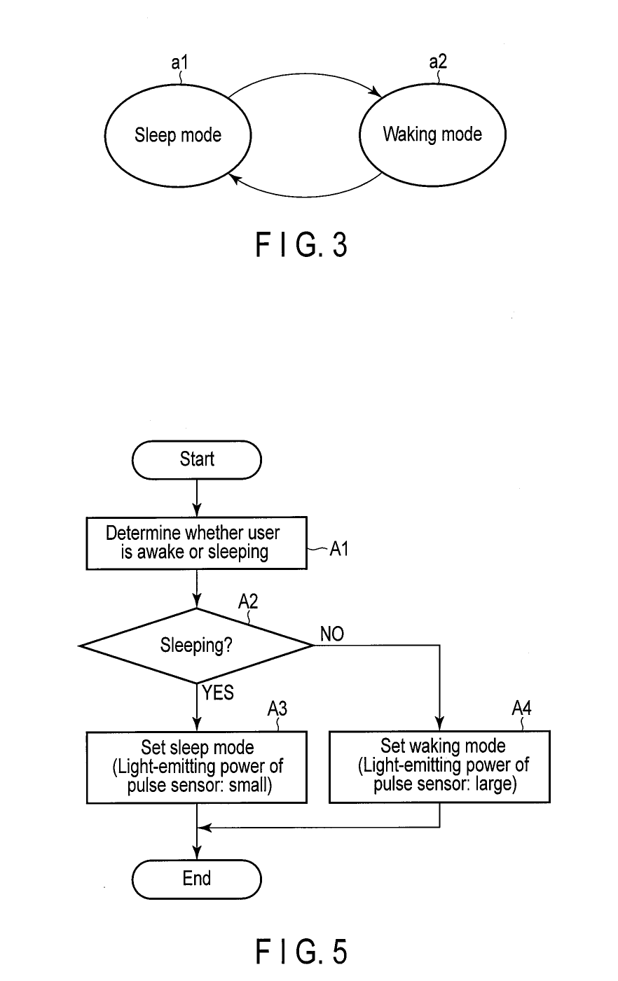

The biometric information acquisition program 100 is configured to set the pulse sensor 35A in a sleep mode if the user wearing the wearable terminal 1 enters a sleeping state, and to set the pulse sensor 35A in a waking mode if the user enters a waking state. That is, as shown in FIG. 3, the state of the pulse sensor 35A in the wearable terminal 1 according to the embodiment transitions adaptively between a sleep mode (a1) and a waking mode (a2). The sleep mode is a mode for causing the light-emitting power of the pulse sensor 35A to be reduced (in comparison with that of the waking mode).

It is assumed that when the user (wearing the wearable terminal 1) is awake, the wearable terminal 1 operates in an environment in which body motion and extraneous light are large. Body motion and extraneous light work as noise in the pulse sensor 35A, which is a photoelectric sensor. On the other hand, it is assumed that when the user is sleeping, the wearable terminal 1 operates in an environment in which body motion and extraneous light are small. Thus, the wearable terminal 1 according to the embodiment is configured to, at the time of awakening when body motion and extraneous light have a great influence, increase the emitting-power of the pulse sensor 35A to a certain extent to secure a signal-to-noise ratio above a standard; and to, at the time of sleep when body motion and extraneous light have a small influence, reduce the emitting-power of the pulse sensor 35A (within a range in which a signal-to-noise ratio above a standard can be secured) to restrain the power consumption of the pulse sensor 35A.

For example, since a pulse rate tends to decline in a sleeping state as compared with that in a waking state, whether the user wearing the wearable terminal 1 is in a sleeping state or a waking state can be determined on the basis of a detected value of the pulse sensor 35A. Also, for example, since a specific pattern tends to appear in the movement of an arm in a sleeping state, determination can be made on the basis of a detected value of the acceleration sensor 35B. As a matter of course, determination can also be made complexly on the basis of both of a detected value of the pulse sensor 35A and a detected value of the acceleration sensor 35B. In addition, for example, since body temperature (surface temperature of the human body) tends to decline in a sleeping state as compared with that in a waking state, a detected value of a temperature sensor may also be used. Moreover, assuming that the user in a sleeping state is in an environment of small extraneous light, a detected value of an illuminance sensor can also be used secondarily.

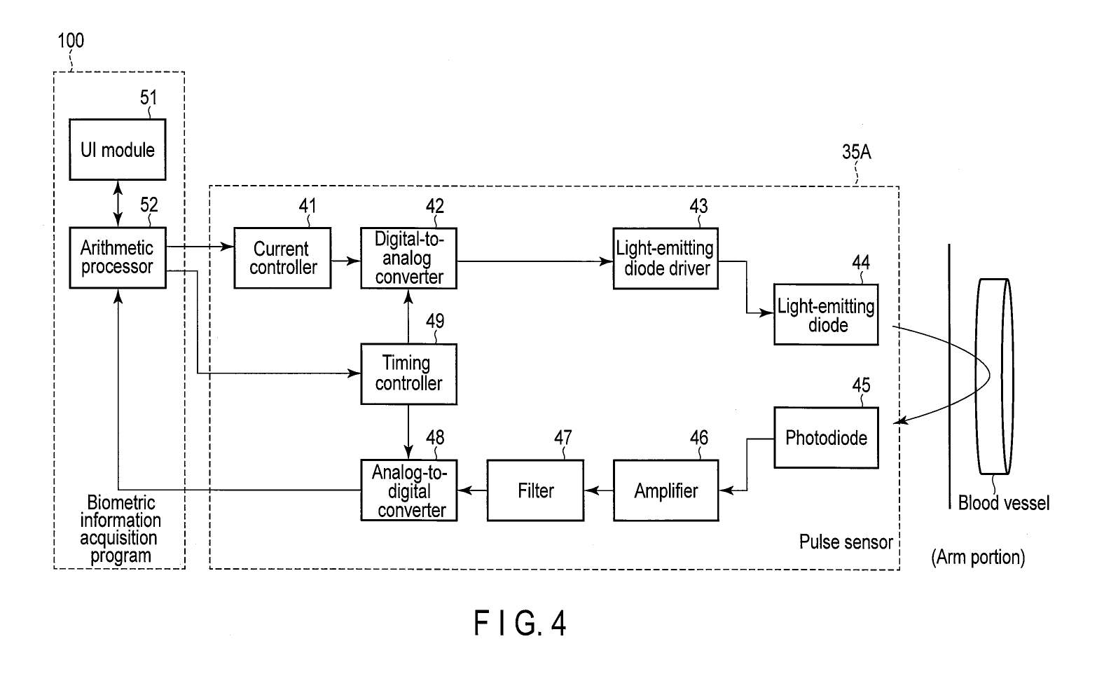

FIG. 4 is an exemplary functional block diagram related to reduction in power consumption by the pulse sensor 35A of the wearable terminal 1. Here, the case of determining whether the user wearing the wearable terminal 1 is in a sleeping state or a waking state on the basis of a detected value of the pulse sensor 35A is assumed.

As shown in FIG. 4, the pulse sensor 35A includes a current controller 41, a digital-to-analog converter 42, a light-emitting diode driver 43, a light-emitting diode 44, a photodiode 45, an amplifier 46, a filter 47, an analog-to-digital converter 48 and a timing controller 49.

The light-emitting diode 44 and the photodiode 45 are disposed on the back surface of the main body 11 adjacent to the skin of the user wearing the wearable terminal 1. The pulse sensor 35A is configured to emit light to a blood vessel close to the skin from the light-emitting diode 44, and to receive its reflected light by the photodiode 45. The light-emitting diode driver 43 is configured to drive the light-emitting diode 44 on the basis of a driving signal supplied from the digital-to-analog converter 42. Accordingly, the light-emitting power of the light-emitting diode 44 can be controlled by controlling the digital-to-analog converter 42 to control a value of the driving signal. Thus, the light-emitting power of the light-emitting diode 44 is controlled by one or both of (a) setting a current value by the current controller 41 and (b) setting a duty ratio by the timing controller 49.

Data indicating the amount of received reflected light is output from the photodiode 45, and is amplified by the amplifier 46. Amplified data is supplied through the filter 47 to the analog-to-digital converter 48, and data (pulse data) is output from the analog-to-digital converter 48 with a timing corresponding to a light-emitting timing of the light-emitting diode 44 on the basis of a synchronizing signal from the timing controller 49.

The biometric information acquisition program 100 includes a user interface (UI) module 51 and an arithmetic processor 52. The arithmetic processor 52 is configured to determine whether the user wearing the wearable terminal 1 is in a sleeping state or a waking state based on pulse data output from the analog-to-digital converter 48 of the pulse sensor 35A.

If it is determined that the user wearing the wearable terminal 1 is in a sleeping state, the arithmetic processor 52 sets the pulse sensor 35A in the sleep mode. More specifically, the arithmetic processor 52 instructs the current controller 41 of the pulse sensor 35A to set a current value of electric power supplied for driving the light-emitting diode 44 low, or instructs the timing controller 49 of the pulse sensor 35A to set a duty ratio which is a proportion of a light-emitting period per unit time of the light-emitting diode 44 low. Both of an instruction to the current controller 41 and an instruction to the timing controller 49 may be given. Thereby, the light-emitting power of the light-emitting diode 44 becomes small and the power consumption of the pulse sensor 35A is reduced.

On the other hand, if it is determined that the user wearing the wearable terminal 1 in a waking state, the arithmetic processor 52 sets the pulse sensor 35A in the waking mode. More specifically, the arithmetic processor 52 instructs the current controller 41 of the pulse sensor 35A to set a current value of electric power supplied for driving the light-emitting diode 44 high (at a reference value), or instructs the timing controller 49 of the pulse sensor 35A to set a duty ratio which is a proportion of a light-emitting period per unit time of the light-emitting diode 44 high (at a reference value). As in the above-described sleep mode, both of an instruction to the current controller 41 and an instruction to the timing controller 49 may be given. Thereby, the light-emitting power of the light-emitting diode 44 becomes large and a signal-to-noise ratio above a standard is secured even in an environment in which body motion and extraneous light are large.

In this way, in the wearable terminal 1 according to the embodiment, the power consumption during sleeping hours which accounts for about one third to one forth of a day can be restrained, and the duration of the battery 37 can be lengthened. Also, the light amount of light escaping through a gap between the back surface of the main body 11 where the light-emitting diode 44 is disposed and the skin of an arm portion of the user (wearing the wearable terminal 1) adjacent to the back surface of the main body 11 can be reduced, and interruption of sleep due to a dazzle of light can be reduced.

In addition, switching of the pulse sensor 35A between the sleep mode and the waking mode can be executed also by an instruction from the user by an operation of the operation button 13. To receive the instruction from the user, the biometric information acquisition program 100 includes the user interface (UI) module 51. The UI module 51 also has the function of displaying biometric information such as a pulse and an active state of an autonomic nerve of the user acquired by using the pulse sensor 35A on the display 12. By means of the UI module 51, the biometric information acquisition program 100 displays, for example, at the time of awakening, a degree of motion intensity based on a pulse rate or a degree of relaxation based on an active state of an autonomic nerve on the display 12. Also, for example, at the time of sleeping, the biometric information acquisition program 100 displays a degree of depth of sleep based on an active state of an autonomic nerve on the display 12.

FIG. 5 is an exemplary flowchart showing a procedure of processing of reduction in power consumption by the pulse sensor 35A executed by the wearable terminal 1.

The wearable terminal 1 determines whether the user wearing the wearable terminal 1 is in a waking state or a sleeping state on the basis of a detected value of at least one sensor of the plurality of sensors 35A, 35B . . . (block A1).

If it is determined that the user is in a sleeping state (YES in block A2), the wearable terminal 1 sets the pulse sensor 35A of the plurality of sensors 35A, 35B . . . in the sleep mode (block A3). More specifically, the light-emitting power of the pulse sensor is reduced. On the other hand, if it is determined that the user is in a waking state (NO in block A2), the wearable terminal 1 sets the pulse sensor 35A in the waking mode (block A4). More specifically, the light-emitting power of the pulse sensor is increased.

As described above, in the wearable terminal 1 according to the embodiment, power consumption can be appropriately reduced in accordance with circumstances.

Moreover, although an example of switching the operation mode of the pulse sensor 35A between the waking mode and the sleep mode in accordance with whether the user wearing the wearable terminal 1 is in a waking state or a sleeping state, more specifically, an example of switching the light-emitting power has been described in the above description, this technique can be applied to not only the pulse sensor 35A but various sensors. By switching a dynamic range of various sensors, for example, setting a mode of outputting a detected value at 16 bits during awakening and setting a mode of outputting a detected value at 8 bits during sleeping, their power consumption can be adaptively reduced. Furthermore, this technique can be applied to not only a sensor, and can also be applied to, for example, control of the operation mode of the whole wearable terminal 1. For example, assuming that biometric information is little in a sleeping state, it is conceivable to lengthen a cycle of sensing. In this case, the loads of an arithmetic processor configured to process sensing data, a memory configured to store sensing data, etc., can be reduced, and thereby reduction in power consumption can be achieved.

Various processes of the present embodiment can be implemented by a computer program. Thus, the same advantages as those of the present embodiment can be easily achieved simply by installing and executing the computer program on a normal computer through a computer-readable storage medium storing the computer program.

The various modules of the systems described herein can be implemented as software applications, hardware and/or software modules, or components on one or more computers, such as servers. While the various modules are illustrated separately, they may share some or all of the same underlying logic or code.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An electronic apparatus wearable by a user, the apparatus comprising:
one or more sensors;
a processor configured to determine whether the user is in a waking state or a sleeping state by using a detected value of at least one sensor of the one or more sensors; and
a controller configured to set an operation mode of the apparatus in a first apparatus mode if it is determined that the user is in a waking state, and to set the operation mode of the apparatus in a second apparatus mode if it is determined that the user is in a sleeping state.

2. The apparatus of claim 1, wherein the controller is configured to set an operation mode of a first sensor of the one or more sensors in a first sensor mode if it is determined that the user is in a waking state, and to set an operation mode of the first sensor in a second sensor mode if it is determined that the user is in a sleeping state.

3. The apparatus of claim 2, wherein:
the first sensor comprises a photoelectric pulse sensor;
the first sensor mode comprises a mode for causing a light-emitting diode of the photoelectric pulse sensor to emit a first light-emitting amount of light; and
the second sensor mode comprises a mode for causing the light-emitting diode to emit a second light-emitting amount of light, the second light-emitting amount being smaller than the first light-emitting amount.

4. The apparatus of claim 3, wherein the controller is configured to control a current value of electric power supplied for driving the light-emitting diode.

5. The apparatus of claim 3, wherein the controller is configured to control a duty ratio of a light-emitting period per unit time of the light-emitting diode.

6. The apparatus of claim 2, further comprising a battery, wherein the first sensor is configured to operate by electric power from the battery.

7. A method of an electronic apparatus wearable by a user, the method comprising:
determining whether the user is in a waking state or a sleeping state by using a detected value of at least one sensor of one or more sensors; and
setting an operation mode of the apparatus in a first apparatus mode if it is determined that the user is in a waking state, and setting an operation mode of the apparatus in a second apparatus mode if it is determined that the user is in a sleeping state.

8. The method of claim 7, wherein the setting the operation mode comprises setting an operation mode of a first sensor of the one or more sensors in a first sensor mode if it is determined that the user is in a waking state, and setting an operation mode of the first sensor in a second sensor mode if it is determined that the user is in a sleeping state.

9. The method of claim 8, wherein:
the first sensor comprises a photoelectric pulse sensor;
the first sensor mode comprises a mode for causing a light-emitting diode of the photoelectric pulse sensor to emit a first light-emitting amount of light; and
the second sensor mode comprises a mode for causing the light-emitting diode to emit a second light-emitting amount of light, the second light-emitting amount being smaller than the first light-emitting amount.

10. The method of claim 9, wherein the setting the operation mode comprises controlling a current value of electric power supplied for driving the light-emitting diode.

11. The method of claim 9, wherein the setting the operation mode comprises controlling a duty ratio of a light-emitting period per unit time of the light-emitting diode.

12. The method of claim 8, wherein:
the apparatus comprises a battery; and
the first sensor is configured to operate by electric power from the battery.

13. A computer-readable, non-transitory storage medium having stored thereon a computer program which is executable by a computer wearable by a user, the computer program controlling the computer to function as:
a processor configured to determine whether the user is in a waking state or a sleeping state by using a detected value of at least one sensor of one or more 2.0 sensors; and
a controller configured to set an operation mode of the computer in a first computer mode if it is determined that the user is in a waking state, and to set an operation mode of the computer in a second computer mode if it is determined that the user is in a sleeping state.

14. The medium of claim 13, wherein the controller is configured to set an operation mode of a first sensor of the one or more sensors in a first sensor mode if it is determined that the user is in a waking state, and to set an operation mode of the first sensor in a second sensor mode if it is determined that the user is in a sleeping state.

15. The medium of claim 14, wherein:
the first sensor comprises a photoelectric pulse sensor;
the first sensor mode comprises a mode for causing a light-emitting diode of the photoelectric pulse sensor to emit a first light-emitting amount of light; and
the second sensor mode comprises a mode for causing the light-emitting diode to emit a second light-emitting amount of light, the second light-emitting amount being smaller than the first light-emitting amount.

16. The medium of claim 15, wherein the controller is configured to control a current value of electric power supplied for driving the light-emitting diode.

17. The medium of claim 15, wherein the controller is configured to control a duty ratio of a light-emitting period per unit time of the light-emitting diode.

18. The medium of claim 15, wherein:
the computer comprises a battery; and
the first sensor is configured to operate by electric power from the battery.
